(19) United States
(12) Patent Application Publication
(10) Pub. No.: US 2002/0136768 A1
(43) Pub. Date: Sep. 26, 2002

(54) Antimicrobial composition

(76) Inventor: Victor J. Staats, (US)

Correspondence Address: Robert M. Downey, ROBERT M. DOWNEY, P.A., Suite 350, 150 E. Palmetto Park Road, Boca Raton, FL, 33432, US

(21) Appl. No.: 10/010,111

(22) Filed: Nov. 13, 2001

Related U.S. Application Data
(60) Provisional application No. 60/248,381, filed on Nov. 14, 2000.

(51) Int. Cl. A61K 9/14; A61K 31/14

(52) U.S. Cl. 424/484; 514/642; 514/643

(57) ABSTRACT

A composition for a hand wash provides antiviral, antibacterial, and antifungal properties and includes an effective amount of one or more quaternary ammonium compounds, surfactants, and a hydrophilic film forming agent in a functional complex. When applied to the skin, the composition kills most common household germs within 30 seconds and provides persistent protection against potentially harmful, disease-causing pathogens.

### BACKGROUND OF THE INVENTION

1. Field of the Invention

The present invention relates to an antimicrobial composition and, more particularly, to a composition for a hand wash having antiviral, antibacterial, and antifungal properties. The hand wash is applied to the skin to protect against potentially harmful, disease-causing pathogens.

2. Discussion of the Related Art

Each day, the population is exposed to harmful and destructive organisms which come in contact with the skin. There is increasing concern that exposure to many of these disease-causing pathogens presents a serious threat to the health and well being of the general public. For instance, staph and e-coli are often spread by cross-contamination, wherein a healthy person touches an infected person or surfaces that have come in contact with the bacteria. In the healthcare industry, both healthcare workers and patients are regularly exposed to potentially harmful viruses and bacteria including AIDS and Hepatitis. Recent estimates reveal that over 88,000 people have died from hospital acquired infections last year in the United States alone.

The art is crowded with antibacterial products, and particularly antibacterial soaps and hand wash products. Many of these products contain Triclosan, which has recently come under scrutiny for its ineffectiveness in killing germs. Also, many ordinary hand sanitizers presently on the market contain 60% or more ethyl alcohol, which causes dry skin and evaporates quickly, leaving the hands dry and unprotected shortly after application of the sanitizer.

Presently available antibacterial products, and particularly those containing Triclosan, are ineffective in killing all pathogens on the skin. Moreover, many of the antimicrobial products presently on the market fail to provide long lasting protection. Thus, if a person comes into contact with disease-causing pathogens shortly after application of one of these products, there is a possibility that the pathogens will not be killed.

The composition of the present invention provides a highly effective antibacterial hand wash which has been clinically proven to kill 99.99% of most common household germs in as little as 30 seconds, without water. Moreover, the composition has been proven to kill germs for up to four hours after application. In clinical testing, the composition of the present invention killed over 98% of staph germs four hours after initial application. And, unlike products containing high amounts of ethyl alcohol, the composition of the present invention contains superior moisturizers which leave the user's skin feeling soft, smooth and clean for hours after application.

### SUMMARY OF THE INVENTION

The present invention provides a composition for topical application to the skin and, more particularly, a hand wash applied to the skin to provide persistent antiviral, antibacterial, and antifungal protection. In a preferred embodiment, the composition comprises one or more quaternary ammonium compounds, a hydrophilic film forming agent, a hydrophobic waterproofing agent, surfactants, an inorganic silicate and water. The composition provides protection against harmful and destructive organisms on the hands, feet and general areas of the skin where protection is needed. When applied to the skin, the composition provides a means to combat a broad spectrum of organisms that can cause contamination and cross-contamination.

### OBJECTS AND ADVANTAGES OF THE INVENTION

It is a primary object of the present invention to provide a safe and effective hand wash for providing long lasting and persistent antiviral, antibacterial, and antifungal protection.

It is a further object of the present invention to provide a composition for a hand wash which protects against potentially harmful and destructive organisms.

It is still a further object of the present invention to provide a composition for a hand wash which provides persistent protection against harmful and destructive organisms and which aids in the healing process of damaged infected skin.

It is still a further object of the present invention to provide for a composition for a hand wash which provides a therapeutic moisturizer.

It is still a further object of the present invention to provide a composition for a hand wash which sanitizes the skin within 60 seconds after application and which remains on the skin with long lasting, persistent protection against potentially harmful and destructive organisms.

It is still a further object of the present invention to provide a composition for a hand wash, as set forth above, which is effective with or without a potable rinse.

It is yet a further object of the present invention to provide a composition for a hand wash which provides antiviral, antibacterial, and antifungal properties and which is further waterproof and resistant to oil and salt.

### DETAILED DESCRIPTION OF THE PREFERRED EMBODIMENT

The present invention is directed to an antiviral, antibacterial, and antifungal composition for use in a hand wash product to provide long lasting, persistent protection against harmful and destructive organisms.

In a preferred embodiment, one or more quaternary ammonium compounds are contemplated for use as biologically active functional substances. For instance, the following quaternary ammonium compounds are known to be biocidally effective as a functional substance in the composition of the present invention:

benzalkonium chloride, known commercially as BTC 50;

benzathonium chloride;

dimethyl benzyl ammonium chloride blended with dimethyl ethylbenzyl ammonium chloride, known commercially as BTC 2125M.

A hydrophilic swellable film forming compound is provided in the composition. In a preferred embodiment, a cross-linked acrylic acid polymer, such as Carbopol 940, manufactured by B.F. Goodrich, is selected. The acrylic acid polymer is in a mixture with an inorganic silicate which is water insoluble. The inorganic silicate, such as Laponite XLG, distributed by Laparte, Inc., provides a support mechanism for the acrylic acid polymer.

Examples of other polymers which are contemplated for use as the hydrophilic film forming agent include the following:

Carbopol 934;

Carbopol 941;

Carbopol 980;

Carbopol 981;

Carbopol 5984;

Carbopol 2001;

Carbopol 2020;

Carbopol 2050;

Pemulen TR1;

Pemulen TR2;

Avalure 1342;

Avalure 1382; and

Avalure 2984.

The composition further contains a hydrophobic waterproofing agent. The hydrophobic waterproofing agent is preferably a polymer derived from vinylpyrrolidone and a long chain alpha olefin. In a preferred embodiment, the hydrophobic waterproofing agent is a PVP/eicosene copolymer (polyvinylpyrrolidone and eicosene). The product known as Ganax V 220, manufactured by GAF Chemicals Corp., is an example of a hydrophobic waterproofing agent in accordance with the preferred embodiment.

The incorporation of various surfactants is contemplated within the scope of the present invention. Specifically, the composition includes one or more surfactants selected from the following group:

Tergitol;

Triton;

Makon;

Triethanolamine;

AMP 95.

The following is an example of the formula of the composition of the present invention:

### EXAMPLE 1

| Ingredient Name | Range |
|---|---|
| Quaternary ammonium compound | 50 ppm-2000 ppm |
| (for example: Benzalkonium Chloride) | |
| Copolymer (for example: 940) | .01% to 15% |
| Inorganic silicate (for example: Laponite) | .01% to 15% |
| Hydrophobic waterproofing agent | .025% to 15% |
| (for example: Ganax V 220) | |
| Surfactants (for example: Triethanolamine) | .05% to 20% |

Purified water to 100 percent of specific formula

Actual percentages for each formula will vary depending upon the need of the composition and the particular areas of application to the skin (e.g., hands, feet, intact skin, damaged skin, infected skin, mouth or skin covered by mucus membranes, etc.).

The preparation of the composition of the present invention is accomplished in accordance with following procedure:

Mixing Instructions:

1. In a stainless steel mixing tank, add one-half of the amount of purified water. Add the hydrophilic water swellable polymer and mix using a high speed shear mixer. Then heat the mixture to 70° C. Then add the hydrophobic waterproofing agent at a high speed mix. Hold the mixture at 70° C. for 15 minutes.

2. Begin cooling the mixture. Then add the inorganic silicate. Mix until it is in solution, to produce a first mixture.

3. In a separate stainless steel mixing tank, add the remaining one-half of purified water and the quaternary ammonium compound. Mix well. Then add one-half of the surfactants and mix until it is all in solution, producing a second mixture.

4. Add the remaining half of the surfactants to the first mixture at a high speed mix.

5. Combine the two mixtures slowly until they are completely mixed. Then, mix the entire mixture at a high speed until it is all in solution.

### EXAMPLE 2

| Ingredient Name | Range |
|---|---|
| Quaternary ammonium compound | 250 ppm-750 ppm |
| (e.g. dimethyl benzyl ammonium chloride/ | |
| dimethyl ethylbenzyl ammonium chloride) | |
| Copolymer (e.g. Carbopol 934) | 0.5%-1.5% |
| Inorganic silicate (e.g. Laponite XLG) | 0.5%-1.5% |
| Hydrophobic waterproofing agent (e.g. Ganax | 0.05%-0.5% |
| V220) | |
| Surfactants (e.g. Makon 10) | 0.5%-5.0% |
| Catalyst (e.g. Triethanolamine) | 1.0%-5.0% |
| Stabilizer (e.g. 1-3 Butylene Glycol) | 2.0%-7.0% |
| Water to 100% of formula | |

The composition of the above example is prepared in the manner described above with reference to Example 1.

### EXAMPLE 3

| Ingredient Name | Range |
|---|---|
| Quaternary ammonium compound | 50 ppm-1000 ppm |
| (e.g. dimethyl benzyl ammonium chloride/ | |
| dimethyl ethylbenzyl ammonium chloride) | |
| Copolymer (e.g. Carbopol 940) | 0.1%-1.0% |
| Inorganic silicate (e.g. Laponite LXG) | 0.5%-3.0% |
| Hydrophobic waterproofing agent (e.g. Ganax | 0.1%-2.0% |
| V220) | |
| Surfactant (e.g. Tergitol) | 0.5%-1.5% |
| Catalyst (e.g. AMP 95) | 0.5%-4.0% |
| Stabilizer (e.g. 1-3 Butylene Glycol) | 0.5%-1.5% |
| Water to 100% of formula | |

The above composition is prepared according to the procedure set forth above with respect to Example 1.

In each of the above examples, the percentage range of each ingredient is based on the amount of each ingredient by weight of the entire composition.

While the invention has been described in accordance with preferred and practical embodiments thereof, it is recognized that departures from the instant disclosure are contemplated within the spirit and scope of the invention as set forth in the following claims as interpreted under the doctrine of equivalents.

What is claimed is:

1. An antimicrobial composition for topical application to the skin comprising:
a biocidally effective amount of at least one quaternary ammonium compound;
a hydrophilic film-forming agent;
a hydrophobic waterproofing agent;
at least one surfactant; and
water.

2. The composition as recited in claim 1 wherein said at least one quaternary ammonium compound is benzalkonium chloride.

3. The composition as recited in claim 1 wherein said at least one quaternary ammonium compound is benzathonium chloride.

4. The composition as recited in claim 1 wherein said at least one quaternary ammonium compound is a blend of n-alkyl dimethyl benzyl ammonium chloride and n-alkyl dimethyl ethylbenzyl ammonium chloride.

5. The composition as recited in claim 1 further comprising a mixture of two or more quaternary ammonium compounds selected from the group consisting of:
benzalkonium chloride;
benzathonium chloride;
n-alkyl dimethyl benzyl ammonium chloride; and
n-alkyl dimethyl ethylbenzyl ammonium chloride.

6. The composition as recited in claim 1 wherein said hydrophilic film-forming agent is a hydrophilic swellable polymer.

7. The composition as recited in claim 6 wherein said hydrophilic swellable film-forming polymer comprises a cross-linked acrylic acid polymer and an inorganic silicate.

8. The composition as recited in claim 1 wherein said hydrophobic waterproofing agent comprises:
a polymer derived from vinylpyrrolidone and a long chain alpha olefin.

9. An antimicrobial composition for topical application to the skin comprising:
a biocidally effective amount of a functional substance;
a hydrophilic film-forming agent;
a hydrophobic waterproofing agent;
at least one surfactant; and
water.

10. An antimicrobial composition for topical application to the skin comprising:
a biocidally effective amount of at least one quaternary ammonium compound;
a hydrophilic film-forming agent;
a hydrophobic waterproofing agent;
an inorganic silicate;
at least one surfactant; and
water.
